(19) United States
(12) Patent Application Publication
(10) Pub. No.: US 2022/0401152 A1
(43) Pub. Date: Dec. 22, 2022

(54) METHOD FOR VISUAL SUPPORT IN NAVIGATION AND SYSTEM

(71) Applicant: Siemens Healthcare GmbH, Erlangen (DE)

(72) Inventor: Marcus Pfister, Bubenreuth (DE)

(21) Appl. No.: 17/764,973

(22) PCT Filed: Sep. 2, 2020
(86) PCT No.: PCT/EP2020/074378

§ 371 Date: Mar. 29, 2022

(30) Foreign Application Priority Data
Sep. 30, 2019 (DE) 10 2019 215 001.2

(51) Int. Cl. A61B 34/20; A61B 6/12; A61B 6/00

(52) U.S. Cl. CPC A61B 34/20; A61B 6/12; A61B 6/504; A61B 6/4085; A61B 6/5235; A61B 6/54; A61B 6/463; A61B 2034/2051

(57) ABSTRACT

For particularly quick and error-reduced navigation in vessel branches, a method is provided for visual support during navigation of a medical catheter introduced into a hollow organ system of a patient in a hollow organ branch, comprising the following steps: providing an, in particular pre-segmented, volume image of the hollow organ system and the hollow organ branch, which has been captured by means of an X-ray device; providing information relating to the geometric shape of the catheter tip; receiving a current projection image of the catheter tip, in particular by means of a cone beam X-ray device; registering the volume image and the projection image in the event that there is no pre-registration; determining the current position and current orientation of the catheter tip on the projection image based on the projection image; determining the relative position and relative orientation of the catheter tip in relation to the hollow organ branch; and displaying information relating to the determined relative position and/or relative orientation of the catheter tip in relation to the hollow organ branch.

This application is the National Stage of International Application No. PCT/EP2020/074378, filed Sep. 2, 2020, which claims the benefit of German Patent Application No. DE 10 2019 215 001.2, filed Sep. 30, 2019. The entire contents of these documents are hereby incorporated herein by reference.

### BACKGROUND

The present embodiments relate to visual support when navigating a medical catheter.

An abdominal aortic aneurysm 2 (e.g., see FIG. 1) is a vascular bulging at the abdominal aorta 1, the extension of which into the leg arteries is referred to as an iliac aneurysm. This is either treated in an open abdominal operation or in a minimally invasive manner through the insertion of what is known as a stent graft 3. A minimally invasive method of this kind is referred to as endovascular aneurysm repair (EVAR). Introduced into the abdominal aorta 1 via both sides of the groin are guide wires 4 and catheters, via which one or more stent grafts 3 (e.g., combinations of a stent and an artificial blood vessel) are introduced. This often happens under X-ray control (e.g., fluoroscopy). In order to minimize the application of iodine-containing contrast agent (which may potentially be harmful to kidneys) in this context, various methods are known that overlay a two-dimensional fluoroscopy image (e.g., projection image) with registered preoperative datasets (e.g., three-dimensional volume images, such as CT angiographies). FIG. 2 shows a typical fusion image 6 consisting of an overlaying of a volume image 5 and a projection image.

For example, however, the probing of smaller vessel branches (e.g., the renal arteries) is still difficult and time consuming. Indeed, a large number of catheters with various shapes exist as aids for being able to better probe the individual branches depending on the situation, and overlaying the images is also helpful, as the branches are shown permanently for orientation purposes. For successful probing, however, the catheter is not only to be correctly aligned (e.g., correctly rotated) in the image plane, but also in the depth, which sometimes may only be seen to a limited extent in the projection image.

In known methods, the vessel branches are probed by a doctor, for example, using the catheters “by instinct” or according to the “trial and error” method, possibly based on the overlaid additional information. EVAR procedures are performed on angiography systems under fluoroscopic control. To this end, CTs are generally segmented (e.g., in center lines and surface grids), and the planning of EVAR procedures and registration of angiography systems is performed on the basis thereof. In this context, circular or oval markers are placed at vessel branches automatically (e.g., syngo EVAR Guidance).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method that provides support that enables a particularly rapid and error-reduced navigation into vessel branches is provided. As another example, a system suitable for performing the method is provided.

A method according to an embodiment for visual support when navigating a medical catheter that is introduced into a hollow organ system of a patient, into a hollow organ branch is provided. The method includes providing a volume image (or volume image dataset) of the hollow organ system and the hollow organ branch. The volume image is, for example, presegmented and was recorded by an X-ray device. The method includes providing information regarding the geometric shape of the catheter tip, recording a current projection image of the catheter tip (e.g., by a cone beam X-ray device), and registering the volume image and the projection image in the event that no preregistration is present. The method also includes determining the current position and current orientation of the catheter tip on the projection image based on the projection image, determining the relative position and relative orientation of the catheter tip in relation to the hollow organ branch, and indicating an item of information regarding the determined relative position and/or relative orientation of the catheter tip in relation to the hollow organ branch. By the method according to the present embodiments, the doctor performing an intervention is provided with a support for navigating more rapidly and safely through hollow organs and especially into small hollow organ branches, and, therefore, for making the intervention more gentle and with a lower risk of injury for the patient. Through the more rapid navigation, it is possible to save both radiation and contrast agent, and thus further lower the load for the patient. If the projection image is updated by recording a further current projection image, then, accordingly, it is possible to update the determination of the current position and current orientation of the catheter tip on the projection image based on the projection image, the determination of the relative position and relative orientation of the catheter tip in relation to the hollow organ branch, and the indication. This may be repeated as often as desired or required. The method may be advantageous when navigating in vessels such as arteries or veins or in bronchial systems, for example.

In this context, the orientation of an object may be the three-dimensional alignment of the object in space in relation to a coordinate system. In an object that extends substantially in two dimensions, such as a typical partially circular catheter tip (see further below for examples of shapes), for example, it may also make sense to indicate an angle of rotation in relation to an axis (e.g., in the image plane or in parallel therewith). In this context, the relative position and relative orientation of the catheter tip in relation to the hollow organ branch may be the difference or the amount of the difference between the position of the catheter tip and the position of the hollow organ branch and the difference or the amount of the difference between the orientation of the catheter tip and the orientation of the hollow organ branch in relation to the same coordinate system. For the object that extends substantially in two dimensions, such as the typical, partially circular or rounded catheter tip (see below), for example, it is possible to use a relative angle of rotation (e.g., difference between the angle of rotation of the catheter tip and the angle of rotation of the hollow organ branch relative to an axis (in parallel with or in the image plane)).

The volume image involves a 3D dataset relevant to the procedure (e.g., a CT angiography performed before the procedure or a CT recorded during the intervention using a C-arm X-ray device, such as the Siemens syngo DynaCT; with the administration of contrast agent), which shows the significant anatomy of the hollow organs to be deformed (e.g., in EVAR, thus the aorta and/or the iliac arteries). According to one embodiment, the volume image is segmented with regard to the hollow organ system and the hollow organ branch of the hollow organ system (e.g., in the event that no presegmentation is present). A segmentation of this kind is a known method, which considerably simplifies the further processing of 2D or 3D images by identifying certain structures (e.g., the hollow organ) and marks or highlights the structures, for example. Subsequently, the hollow organ segmented in this way may be represented as a grid or center line, for example. If an appropriate presegmentation is already present, then this act may also be omitted.

Additionally, further information regarding the hollow organ branch that has been taken from the volume image or obtained in other ways may already be present. This may be displayed in the form of circular or oval markers (e.g., ostium markers), for example, or shown in the volume image or a fusion image. The markers (e.g., ostium markers) may be placed automatically or manually (e.g., as part of the Siemens product “syngo EVAR Guidance”).

A registration of the volume image and the current projection image is performed in accordance with known registration methods, usually via a 2D-3D or 3D-3D registration. If the volume image to be overlaid was recorded by the same X-ray device as the projection image (e.g., live projection image), then this act of registration may be omitted. If the volume image and the current projection image are registered (e.g., preregistered or registered as part of a registration method), then a fusion image consisting of a volume image and a projection image may be displayed on a display unit, for example. If the projection image is updated by recording a further current projection image, then the fusion image may also be updated, for example, by replacing the first projection image or through additional superimposition.

The information regarding the geometric shape of the catheter tip may, for example, be taken from a database or a memory. The information may be present as a segmented image, for example, or numerically, as a spline, polygonal chain, or the like, for example. The shape of the catheter tip of known catheters used for interventions of this kind usually extends approximately in one plane (e.g., almost two-dimensionally with only a very low extent in the third dimension) and is additionally embodied in a partially rounded or circular manner (e.g., as a spiral (“pigtail”)), with a simple curvature or with a double curvature (e.g., “shepherd hook”). The representation of a catheter of this kind in the projection image changes with an angle of rotation of the catheter tip relative to an axis in the image plane or projection plane.

By overlaying the volume image and the current projection image, it is possible to determine the position of the catheter in two dimensions within the image plane in general via simple geometric calculations, possibly with the inclusion of image recognition and image processing methods. There are a plurality of options for determining or calculating the orientation, which may be used alone or in combination. It is also simply possible for an estimation to take place.

According to one embodiment, the current orientation of the catheter tip is calculated from the projection image using the projected mapping of the catheter tip on the projection image and/or from the beam geometry of the X-ray beam recording the projection image. In many cases (e.g., if the projection does not take place exactly along the two-dimensional shape of the catheter tip; not in the plane of the catheter tip), it is possible to infer from the projected mapping, if the shape of the catheter is known, the orientation of the catheter tip (e.g., partially rounded or circular catheter tip) or the angle of rotation of the catheter tip in relation to the image plane in a very exact manner, or to calculate the orientation and the angle of rotation of the catheter mathematically from the geometric relationships of the projected mapping. Thus, the cosine of the angle of rotation α of the catheter tip about an axis in the image plane or in parallel with the image plane as a relationship between actual width TB and projected width PB of the partially rounded catheter tip may be calculated: cos(α)=(PB)/(TB). The actual width TB of the rounded catheter tip is known; the projected width PB is determined from the current projection image via an image recognition. In addition or as an alternative, in the event that the projection takes place along the shape of the catheter tip, the beam geometry of the X-ray beam recording the projection image may be included (e.g., if a cone beam projection is involved). For a cone beam projection, the projections of the individual beams are different due to asymmetry, providing that the orientation or the angle of rotation of the catheter tip may also be calculated therefrom. Details regarding this are described further below.

According to a further embodiment, the current orientation of the catheter tip is determined from the projection image using a pretrained machine learning algorithm. This may be used as an alternative or in addition to the determination options described above. The machine learning algorithm may be pretrained based on a large number of projection images of catheter tips and the associated orientations.

From the position and/or orientation of the catheter tip and the position and/or orientation of the hollow branch (e.g., which may be determined from the volume image), the relative position and relative orientation of the catheter tip in relation to the hollow organ branch is subsequently determined, and an item of information is indicated to this end. Such an indication of an item of information is possible in many forms. Thus, it is possible for a visual, an acoustic, or a haptic indication to be performed. According to one embodiment, the indication is formed visually by a mapping (e.g., the catheter tip and the hollow organ branch are indicated schematically), a graphic symbol (e.g., arrow(s) or circle(s) that indicate a direction of rotation are shown), a numerical indication (e.g., a degree indication is shown), or a color indication (e.g., the mapping is superimposed with colors, or a traffic light display that is colored red, yellow, or green is indicated depending on the relative angle of rotation). In the event of a change (e.g., after recording a new current projection image), it is also possible for a convergence to be indicated visually or acoustically (e.g., in the form of various sounds or colors in the case of a convergence, such as reduction of the relative angle of rotation).

According to a further embodiment, markers are used and possibly displayed in order to determine the position and orientation of the hollow organ branch. In this context, a known method for determining the position/orientation of vessel branches is involved. Here, the markers are shown permanently in the projection image (e.g., live fluoroscopy image) or the fusion image consisting of an overlaying of the volume image and the current projection image during an intervention (e.g., as circles or ovals, depending on orientation).

The present embodiments also include a system for performing a method for visual support when navigating a medical catheter that is introduced into a hollow organ system of a patient, into a hollow organ branch. The system includes an imaging device for recording projection images, a memory apparatus for storing data and image data, and an image processing apparatus for performing segmentations of medical volume images and/or projection images. The system also includes a computing unit for determining the position and the orientation of the catheter tip and for determining the relative position and relative orientation of the catheter tip in relation to the hollow organ branch, and an indication apparatus for indicating information regarding the determined relative position and/or relative orientation and a system controller for actuating the system. The system may also have a pretrained machine learning algorithm that is embodied to determine the current orientation of the catheter tip from the projection image.

The present embodiments also include a computer program product that includes a program and may be directly loaded into a memory of a programmable computing unit, with program means in order to carry out a method according to the present embodiments when the program is executed in the computing unit.

### BRIEF DESCRIPTION OF THE DRAWINGS

FIG. 1 shows a view of an abdominal aortic aneurysm with an introduced stent according to the prior art;

FIG. 2 shows a view of a 2D-3D overlay according to the prior art;

FIG. 3 shows a view of various typical catheter tips according to the prior art;

FIG. 4 shows a sequence of a method according to an embodiment for visual support when navigating a catheter into a hollow organ branch;

FIG. 5 shows a view of a 2D-3D overlay of volume image and projection image with markers shown for the hollow organ branches;

FIG. 6 shows a view of a catheter in a hollow organ on a projection image;

FIG. 7 shows a view of the catheter in the hollow organ orthogonally to the projection view of the projection image in FIG. 6;

FIGS. 8 to 10 show views of a catheter tip on a projection image according to FIG. 6;

FIGS. 11 to 13 show views of an angle of rotation of the catheter tips of FIGS. 8 to 10 orthogonally to the projection views of the projection image in FIG. 6;

FIG. 14 shows a view of an angle of rotation of a catheter tip and the angle of rotation of the hollow organ branch relative to the image plane;

FIG. 15 shows a view of an indication of an item of information regarding the determined relative orientation; and

FIG. 16 shows a system for performing the method according to an embodiment.

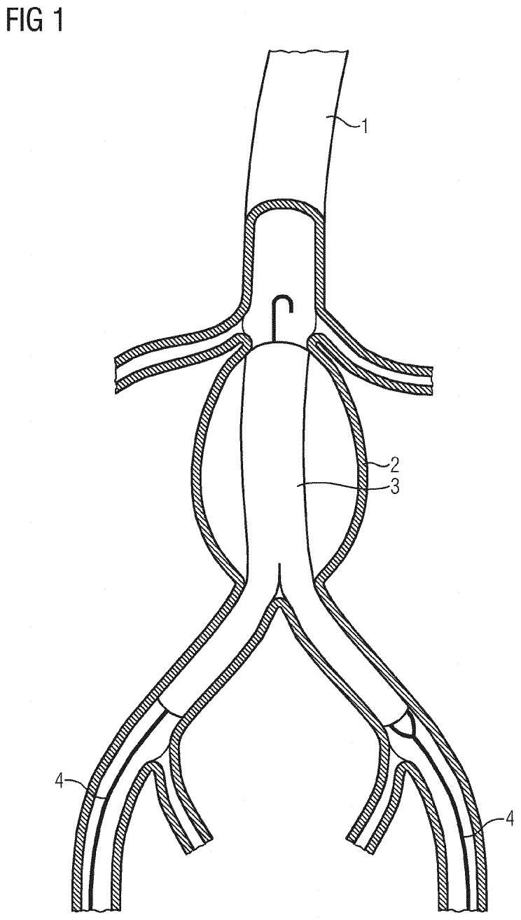

### DETAILED DESCRIPTION

The method according to the present embodiments are advantageous as visual support while navigating a medical catheter that is introduced into a hollow organ system of a patient, into a hollow organ branch. Examples of such navigation are the minimally invasive treatment of aortic aneurysms (EVAR), the replacement of aortic valves, coronary artery interventions, interventions in interventional radiology, or neuroradiology.

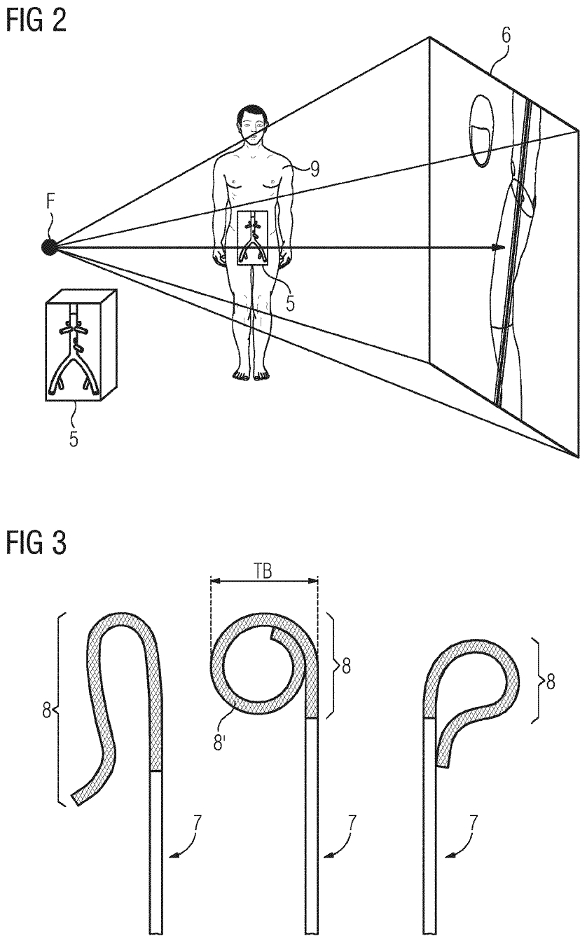

FIG. 3 shows various catheters 7 with typical catheter tips 8 for use in hollow organs (e.g., when probing smaller hollow organ or vessel branches (renal arteries)). The shape of the catheter tip 8 of known catheters 7 usually extends approximately in one plane (e.g., extends two-dimensionally with only a very low extent in the third dimension), and additionally, the catheter tip 8 is embodied in a partially rounded or even circular manner (e.g., as a spiral (“pigtail”, center catheter), with a simple curvature or with a double curvature (e.g., “shepherd hook”, left catheter). The representation of the catheter 7 in the projection image changes with an angle of rotation of the catheter tip relative to an axis in the image plane or projection plane or in parallel with the image plane or the projection plane. By way of example, this is shown for the spiral 8′. If the plane of the spiral 8′ lies entirely in the image plane, then the actual width TB of the spiral 8′ corresponds exactly to the projected width PB of the spiral 8′. In the event of deviations (e.g., if the plane of the spiral 8′ is tilted about the image plane by an angle of rotation α), the projected width PB of the spiral 8′ reduces. The partially round catheter tips are also used for the most gentle possible forward movement within hollow organs. Other shapes of catheter tips, however, may also be used as part of the method according to the present embodiments. These may, however, have an extent in at least two dimensions.

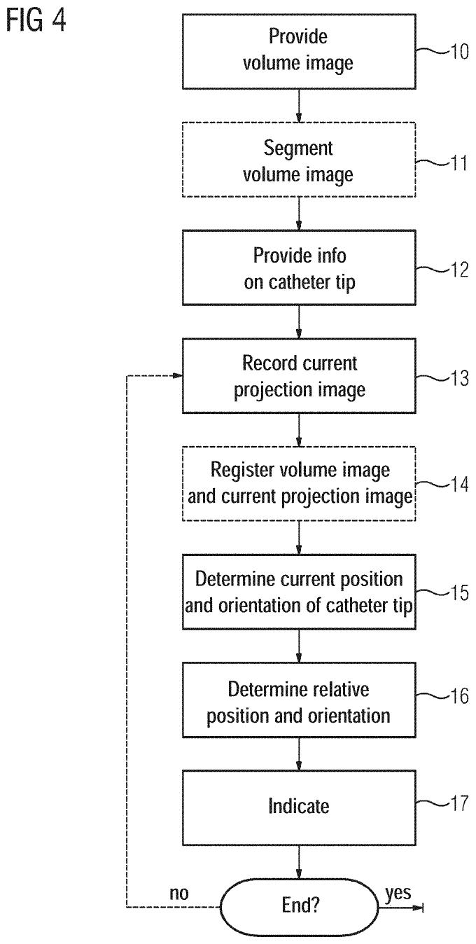

FIG. 4 shows acts of the method. In a first act 10, a volume image or a three-dimensional (3D) dataset of the hollow organ system and the hollow organ branch is provided. The volume image was recorded by an X-ray device. The volume image may, for example, be retrieved from a memory unit or from a database and/or transmitted from a communication unit. The volume image may also be recorded and provided directly. The volume image or the 3D dataset may, for example, be formed of a CT angiography or a C-arm CT (e.g., with the administration of contrast agent). For example, as part of an EVAR procedure, the volume image may map part of the aorta and/or the iliac arteries.

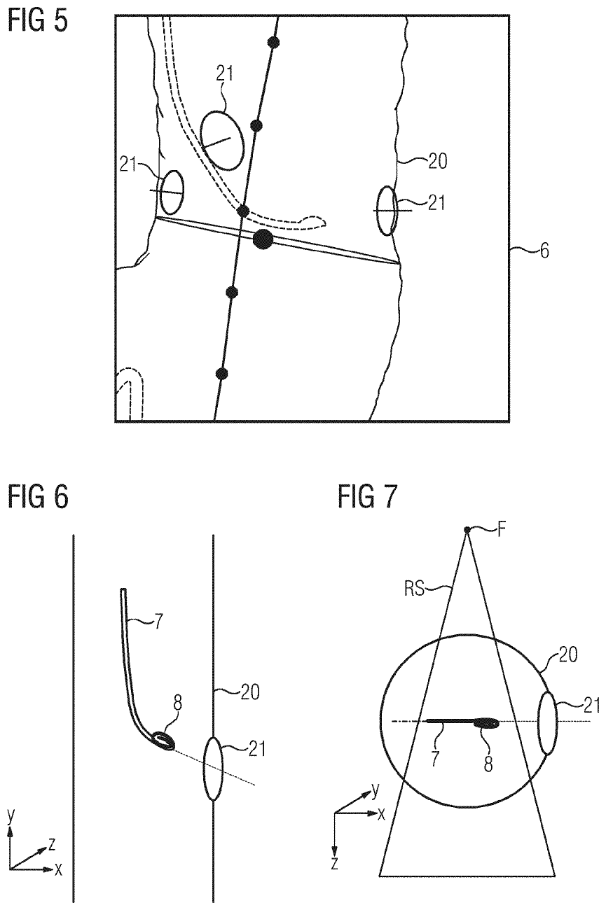

The volume image may already be presegmented (e.g., may have already been segmented automatically or by a user in a preceding act). If this is not the case, then a segmentation of the volume image (e.g., with regard to the hollow organs and hollow organ branches) takes place in an optional second act 11. Segmentation methods are generally known and are often used for the recognition of medical structures. To perform the segmentation, an image processing unit may be used, for example. A segmentation results in segmented medical structures. Additionally, information regarding the hollow organ branch that has been taken from the volume image or obtained in other ways may already be present. Thus, for example, the hollow organ branches of the hollow organ 20 may be drawn in or shown in the volume image in the form of circular or oval markers 21 (e.g., see FIG. 5). The markers 21 may be placed automatically or manually (e.g., as part of the Siemens product “syngo EVAR Guidance”).

In a third act 12, information regarding the geometric shape of the catheter tip is provided. The information may, for example, be taken from a database or a memory unit and/or transmitted from a communication unit. The information may be present as a segmented image (e.g., numerically), as a spline, as a polygonal chain or the like, for example, or in any other possible form.

In a fourth act 13, a current projection image of the catheter tip is recorded (e.g., by a cone beam X-ray device). Thus, for monitoring the procedure, a projection image or a plurality of projection images (e.g., at regular temporal intervals) are often recorded “live”, taking place before and/or during an intervention. Fluoroscopic X-ray images of this kind are created using, for example, a C-arm X-ray device (e.g., a mobile or permanently installed C-arm X-ray device). A schematic view of one such projection image is shown in FIG. 6. FIG. 6 shows a hollow organ 20 with the catheter 7 situated therein, the catheter tip 8, and a hollow organ branch 21 (e.g., shown using markers for improved visibility). In order to illustrate the geometric relationships, a three-dimensional coordinate system with a first axis x, a second axis y, and a third axis z is shown. The image or projection plane is shown from the first axis x and the second axis y (e.g., the main beam of the X-ray radiation RS projects in the direction of the third axis z). FIG. 7 shows the geometry of the arrangement from a direction orthogonal to the image plane, looking “into the hollow organ 20” on a plane that is formed by the third axis z and the first axis x. The path of the X-ray radiation RS is likewise indicated.

In an optional fifth act 14, the current projection image and the provided volume image are registered with one another (e.g., for the event in which no preregistration is present). A registration of the volume image and the current projection image is performed in accordance with known registration methods, usually via a 2D-3D (or 3D-3D) registration. If the volume image to be overlaid was recorded by the same X-ray device as the projection image or a preregistration is already present, then this act of registration may be omitted. If the volume image and the current projection image are registered, then a fusion image consisting of volume image and projection image may be displayed on a display unit, for example (e.g., see FIG. 2).

In a sixth act 15, the current position and current orientation of the catheter tip on the projection image are determined based on the projection image. The general position of the catheter tip in two dimensions within the image plane may be performed in general via simple geometric calculations, possibly with the inclusion of image recognition and image processing methods. The orientation of the catheter tip, if the geometric shape of the catheter tip is known, in many cases may be determined from the projection of the shape of the catheter tip on the projection image (e.g., by calculation or estimation, with the inclusion of image recognition and image processing methods, which takes the geometric lengths and widths (the projected width PB of the catheter tip) from the projection image). Thus, for a typical catheter tip that has an extent in one plane, such as a spiral or curvature, for example, the determination of an angle of rotation relative to an axis (e.g., to the first axis x) in the image plane (or in parallel therewith) may be sufficient to determine the orientation.

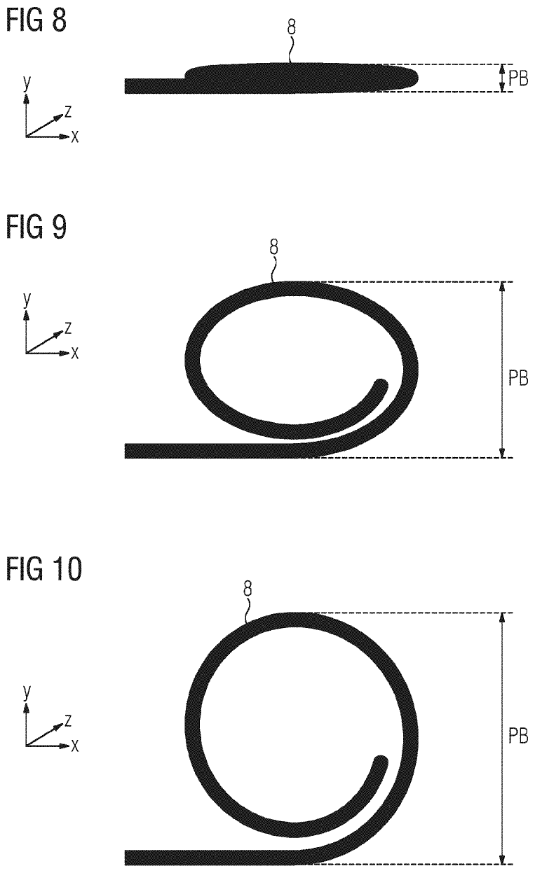

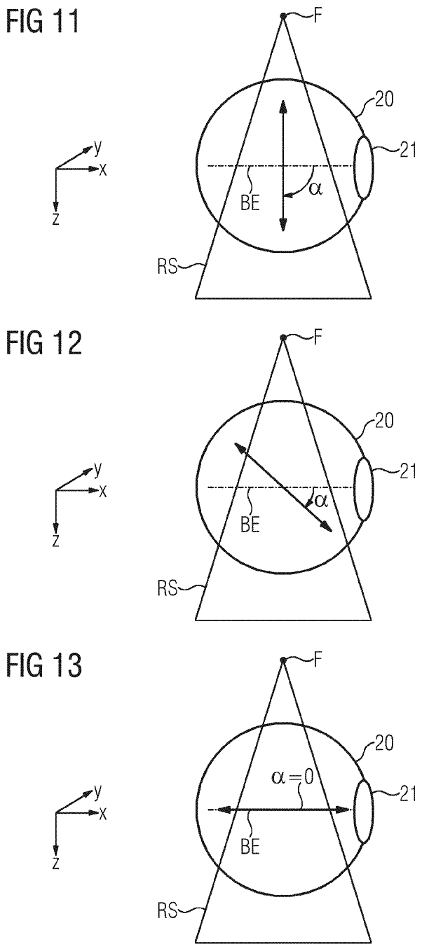

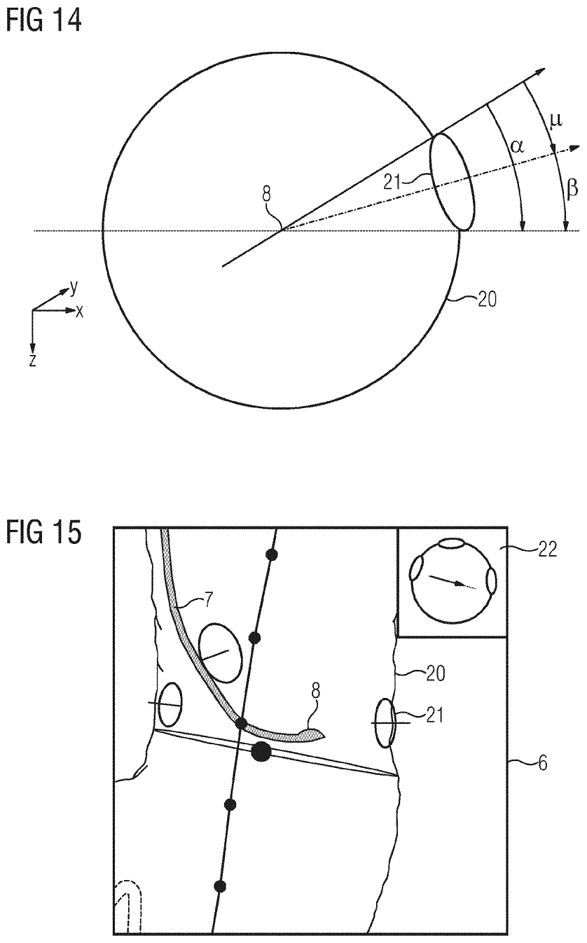

Thus, the cosine of the angle of rotation α of the catheter tip about an axis in the image plane or in parallel with the image plane as a relationship between actual width TB and projected width PB of the partially rounded catheter tip may be calculated: cos(α)=(PB)/(TB). The actual width TB of the rounded catheter tip is known. The projected width PB is determined from the current projection image via an image recognition. FIGS. 8 to 10 show exemplary projected widths PB of a spiral-shaped catheter tip 8, together with the coordinate system. Corresponding views into the hollow organ with the associated angles of rotation a are shown for illustration purposes in FIGS. 11 to 13. In this context, a projection as shown in FIG. 8, with very small projected width PB of the spiral, corresponds to an angle of rotation a as shown in FIG. 11 (e.g., close to 90°). A projection as shown in FIG. 10, with a projected width PB, which is approximately equal to the actual width TB, corresponds to an angle of rotation of approximately 0°, as shown in FIG. 13. A projection as shown in FIG. 9, with a projected width PB, which is approximately ⅓ of the actual width TB, corresponds to an angle of rotation of approximately 30°, as shown in FIG. 12. For illustration purposes, the hollow organ branch lies in the image plane in FIGS. 11 to 13. This is not the case in the normal scenario, however, and the hollow organ branch itself has an angle of rotation b relative to the image plane (see FIG. 14). This is described in further detail below.

In the event that the projected width PB of the catheter tip is very small, it may be necessary to include the beam geometry of the X-ray radiation RS recording the projection image (e.g., if a cone beam projection is involved). For a cone beam projection, the projections of the individual beams are different due to asymmetry, providing that the orientation or the angle of rotation of the catheter tip may also be calculated therefrom.

As an alternative or in addition to the calculation or estimation according to the method described above, the current orientation of the catheter tip is determined from the projection image using a pretrained machine learning algorithm. The machine learning algorithm may have been pretrained based on a large number of projection images of catheter tips and the associated orientations and made available as part of the method. The machine learning algorithm is based on neural networks, for example; a deep learning algorithm may be used, for example.

The use of machine learning algorithms may be used if it is difficult to resolve the geometric relationships with normal image recognition methods. Conventional X-ray devices that are used in minimally invasive interventions (e.g., angiography systems) do not work using parallel radiation, but rather using cone radiation. For a cone beam projection, the projections of the individual beams are different due to asymmetry, providing that the orientation or the angle of rotation of the catheter tip may also be calculated therefrom if the projected width of the catheter tip is very small. Via machine learning methods, it is possible for inaccuracies to be better identified by the deviation from the parallel geometry, but also for ambiguities to be resolved in the event of symmetrical angles of rotation. Such an assignment may be trained in a simple manner by a large number of projection images of corresponding catheter tips being produced and the algorithm learning to assign the respective projection image the angle of rotation α relative to the image plane.

In a seventh act 16, the relative position and/or relative orientation of the catheter tip in relation to the hollow organ branch is determined. In this context, the relative position and relative orientation of the catheter tip in relation to the hollow organ branch may be the difference or the amount of the difference between the position of the catheter tip and the position of the hollow organ branch and the difference or the amount of the difference between the orientation of the catheter tip and the orientation of the hollow organ branch in relation to the same coordinate system. The relative position may be determined in a simple manner via the difference between the previously determined positions.

Since the second angle of rotation b of the hollow organ branch to be met relative to the image plane is often not equal to 0, here too a difference is to be formed between the second angle of rotation of the hollow organ branch and the angle of rotation a of the catheter tip, μ=β−α—(e.g., see in FIG. 14). The differential angle μ=β−α corresponds to the amount that the doctor is to rotate the catheter in order to “meet” the hollow organ branch (e.g., ostium in the aorta). The second angle of rotation β of the hollow organ branch (e.g., the marked ostium to be probed) relative to the image plane is generally known or may be taken from the segmentation.

Subsequently, in an eighth act 17, at least one item of information regarding the determined relative position and/or relative orientation of the catheter tip in relation to the hollow organ branch is indicated. For example, the differential angle m may be indicated in order to give the doctor or user support, as it is then easy for the doctor or user to see the angle by which the doctor or user is to rotate the catheter in order to be able to navigate into the hollow organ branch without injury or resistance. Thus, a visual indication, for example, may be shown in the displayed fusion image or shown in an extra window. The two angles of rotation a and b and/or the hollow organ may be indicated via schematic representation, for example. FIG. 15 shows an exemplary display in which, in an extra display window 22, which is superimposed on the fusion image 6, the angle of rotation a is shown relative to the hollow organ branch, symbolized by an arrow, in a view orthogonal to the fusion image. A numerical indication of the angles of rotation a and b and/or the differential angle of rotation m may also take place in a simple manner. The angles of rotation a and b and/or the differential angle of rotation m may also be shown by a color code in the fusion image (e.g., by the catheter tip and the marker indicating the hollow organ branch being colored depending on how large the corresponding angles of rotation are). The coloration may, for example, be red if the differential angle of rotation is large, yellow if the differential angle of rotation is medium-sized, and green if the differential angle is small. In a similar manner, a traffic light display may simply be present, and/or various sounds may symbolize different differential angles of rotation acoustically. In a simple manner, a graphic symbol, such as an arrow, for example, may also be displayed, which indicates the angle of rotation.

After the eighth act 17, the indication, the method may be terminated if necessary. However, the projection image, for example, may also be updated by recording a further current projection image. Those of the sixth act 15, the seventh act 16, and the eighth act 17 are then likewise repeated in order to show the doctor or the operator an updated indication of an item of information regarding the determined relative position and/or relative orientation of the catheter tip in relation to the hollow organ branch. This updating may be repeated as often as required, if necessary. A helpful support is therefore made available to the doctor or the operator while navigating in a hollow organ, whereby the doctor or the operator is able to navigate into, for example, smaller hollow organ branches in a more rapid and safe manner. If required, the fusion image may also be updated (e.g., by replacing the first projection image or by additional superimposition).

Some of the acts of the method, where sensible, may also be performed in a different order than that disclosed; thus, for example, the information regarding the geometric shape of the catheter tip may be provided first.

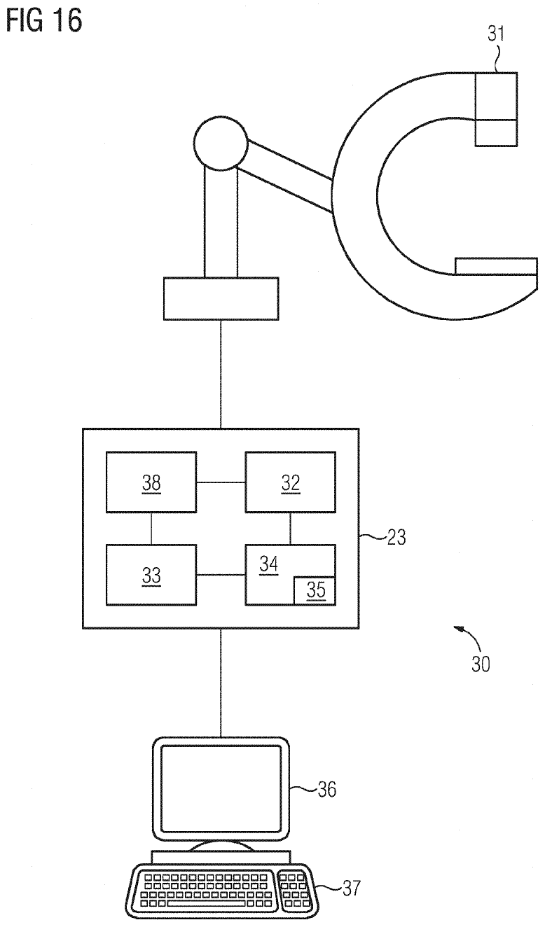

FIG. 16 shows a system 30 for performing the method for visual support when navigating a medical instrument (e.g., catheter) that is introduced into a hollow organ system of a patient, into a hollow organ branch. The system 30 has an imaging device 31, such as an X-ray device, for example, for recording projection images. The imaging device 31 may be formed by a C-arm X-ray device, for example, that is embodied in a mobile or permanently installed manner. The system 30 has a computing unit 34 with a processor. The computing unit 34 is embodied to perform a determination of position and orientation of a catheter tip based on a projection image and a determination of the relative position and relative orientation of the catheter tip in relation to the hollow organ branch. The computing unit may have a pretrained machine learning algorithm 24 that is embodied to determine the current orientation of the catheter tip from the projection image. The machine learning algorithm may be pretrained based on a large number of projection images of catheter tips and the associated orientations. Additionally, the system has a memory unit 32 for storing various image data and information. The system may also have a communication apparatus (not shown) for querying medical data or information from external data memories. The system 30 also includes an image processing apparatus 33 that is embodied to perform segmentations of medical volume images and/or projection images. The system 30 is also assigned a display unit 36 for displaying image data and an input unit 37 for receiving user inputs. The system 30 is actuated by a system controller 38.

The present embodiments include a system for image-based support when navigating instruments, such as catheters, for example, into hollow organs (e.g., helpfully when probing smaller hollow organ branches, such as renal arteries, etc.). An image-based determination (e.g., estimation or calculation) of the rotation of an instrument relative to the image plane, for example, using learning-based methods, a calculation of the rotation of the instrument relative to the vessel branch to be probed, and an indication of the rotation to be performed for the user are provided.

For a particularly rapid and error-reduced navigation into vessel branches, a method for visual support when navigating a medical catheter (e.g., that is introduced into a hollow organ system of a patient) into a hollow organ branch is provided. The method includes providing a volume image that, for example, is presegmented. The volume image is of the hollow organ system and the hollow organ branch that was recorded by an X-ray device. The method includes providing information regarding the geometric shape of the catheter tip, recording a current projection image of the catheter tip (e.g., by a cone beam X-ray device), and registering the volume image and the projection image in the event that no preregistration is present. The method includes determining the current position and current orientation of the catheter tip on the projection image based on the projection image, determining the relative position and relative orientation of the catheter tip in relation to the hollow organ branch, and indicating an item of information regarding the determined relative position and/or relative orientation of the catheter tip in relation to the hollow organ branch.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for visual support when navigating a medical instrument that is introduced into a hollow organ system of a patient, into a hollow organ branch, the method comprising:
providing a volume image of the hollow organ system and the hollow organ branch that was recorded by an X-ray device;
providing information regarding a geometric shape of a tip of the medical instrument;
recording, by a cone beam X-ray device, a current projection image of the tip of the medical instrument;
registering the volume image and the current projection image in the event that no preregistration is present;
determining a current position, a current orientation, or the current position and the current orientation of the tip of the medical instrument on the current projection image based on the current projection image;
determining a relative position, a relative orientation, or the relative position and the relative orientation of the tip of the medical instrument in relation to the hollow organ branch; and
indicating an item of information regarding the determined relative position, the determined relative orientation, or the determined relative position and the determined relative orientation of the tip of the medical instrument in relation to the hollow organ branch.

2. The method of claim 1, wherein the volume image is segmented with regard to the hollow organ system and the hollow organ branch.

3. The method of claim 1, wherein the medical instrument is a catheter, and the tip of the medical instrument is a catheter tip, and
wherein the method further comprises calculating the current orientation of the catheter tip from the current projection image using a projected mapping of the catheter tip on the current projection image, from a beam geometry of an X-ray beam recording the current projection image, or from a combination thereof.

4. The method of claim 3, further comprising determining the current orientation of the catheter tip from the current projection image using a pretrained machine learning algorithm.

5. The method of claim 1, further comprising repeating the determining of the current position, the current orientation, or the current position and the current orientation of the tip of the medical instrument, the determining of the relative position, the relative orientation, or the relative position and the relative orientation of the tip of the medical instrument, and the indicating each time a further current projection image is recorded.

6. The method of claim 1, wherein the medical instrument is a catheter, and the tip of the medical instrument is a catheter tip, and
wherein an indication of the relative orientation of the catheter tip in relation to the hollow organ branch is formed by a mapping, a graphic symbol, a numerical indication, or a color indication.

7. The method of claim 4, wherein the pretrained machine learning algorithm is trained based on a number of projection images of catheter tips and associated orientations.

8. The method of claim 1, wherein the information regarding the geometric shape of the tip of the medical instrument is taken from a database.

9. A system for visual support when navigating a medical catheter that is introduced into a hollow organ system of a patient, into a hollow organ branch, the system comprising:
an imaging device configured to record projection images;
a memory apparatus configured to store data and image data;
an image processing apparatus configured to perform segmentations of medical volume images, the projection images, or the medical volume images and the projection images;
a computing unit configured to:
determine position and orientation of a catheter tip based on a projection image of the projection images; and
determine a relative position and a relative orientation of the catheter tip in relation to the hollow organ branch;
an indication apparatus configured to indicate information regarding the determined relative position, the relative orientation, or the relative position and the relative orientation; and
a system controller configured to actuate the system.

10. The system of claim 9, further comprising a pretrained machine learning algorithm configured to determine a current orientation of the catheter tip from the projection image.

11. (canceled)

12. In a non-transitory computer-readable storage medium that stores instructions executable by one or more processors for visual support when navigating a medical instrument that is introduced into a hollow organ system of a patient, into a hollow organ branch, the instructions comprising:
providing a volume image of the hollow organ system and the hollow organ branch that was recorded by an X-ray device;
providing information regarding a geometric shape of a tip of the medical instrument;
recording, by a cone beam X-ray device, a current projection image of the tip of the medical instrument;
registering the volume image and the current projection image in the event that no preregistration is present;
determining a current position, a current orientation, or the current position and the current orientation of the tip of the medical instrument on the current projection image based on the current projection image;
determining a relative position, a relative orientation, or the relative position and the relative orientation of the tip of the medical instrument in relation to the hollow organ branch; and
indicating an item of information regarding the determined relative position, the determined relative orientation, or the determined relative position and the determined relative orientation of the tip of the medical instrument in relation to the hollow organ branch.

13. The non-transitory computer-readable storage medium of claim 12, wherein the volume image is segmented with regard to the hollow organ system and the hollow organ branch.

14. The non-transitory computer-readable storage medium of claim 12, wherein the medical instrument is a catheter, and the tip of the medical instrument is a catheter tip, and
wherein the instructions further comprise calculating the current orientation of the catheter tip from the current projection image using a projected mapping of the catheter tip on the current projection image, from a beam geometry of an X-ray beam recording the current projection image, or from a combination thereof.

15. The non-transitory computer-readable storage medium of claim 14, wherein the instructions further comprise determining the current orientation of the catheter tip from the current projection image using a pretrained machine learning algorithm.

16. The non-transitory computer-readable storage medium of claim 12, wherein the instructions further comprise repeating the determining of the current position, the current orientation, or the current position and the current orientation of the tip of the medical instrument, the determining of the relative position, the relative orientation, or the relative position and the relative orientation of the tip of the medical instrument, and the indicating each time a further current projection image is recorded.

17. The non-transitory computer-readable storage medium of claim 12, wherein the medical instrument is a catheter, and the tip of the medical instrument is a catheter tip, and
wherein an indication of the relative orientation of the catheter tip in relation to the hollow organ branch is formed by a mapping, a graphic symbol, a numerical indication, or a color indication.

18. The non-transitory computer-readable storage medium of claim 15, wherein the pretrained machine learning algorithm is trained based on number of projection images of catheter tips and associated orientations.

19. The non-transitory computer-readable storage medium of claim 12, wherein the information regarding the geometric shape of the tip of the medical instrument is taken from a database.
